(19) United States
(12) Patent Application Publication
(10) Pub. No.: US 2015/0061166 A1
(43) Pub. Date: Mar. 5, 2015

(54) DEVICE AND METHOD FOR PRODUCING CUSTOM-MADE SPECTACLES

(71) Applicant: LUXEXCEL HOLDING B.V., Goes (NL)

(72) Inventors: Richard Van De Vrie, Wolphaartsdijk (NL); Joris Biskop, Vlissingen (NL)

(21) Appl. No.: 14/388,921

(22) PCT Filed: Mar. 26, 2013
(86) PCT No.: PCT/EP2013/056420

§ 371 Date: Sep. 29, 2014

(30) Foreign Application Priority Data
Apr. 3, 2012 (EP) 12002405.4

(51) Int. Cl. G02C 13/00; B29C 67/00; G02C 7/02; A61B 3/11; A61B 3/032

(52) U.S. Cl. CPC G02C 13/003; G02C 13/005; A61B 3/111; A61B 3/032; G02C 7/027; B29C 67/0059; B33Y 50/00; B33Y 30/00; B33Y 10/00; B29L 2011/0016
USPC 264/1.7; 425/135; 264/1.1

(57) ABSTRACT

The invention relates to a device for producing custom-made spectacles comprising a scanning unit and a producing unit, wherein the scanning unit is configured for scanning at least a part of a customer's face and wherein the producing unit comprises at least a printing device for printing a spectacle lens and/or a spectacle frame, wherein the printing device is configured for printing the spectacle lens and/or the spectacle frame in dependency of scanning data of the scanning unit.

### BACKGROUND

The present invention relates to a device and a method for producing custom-made spectacles. When a customer would like to buy a custom-made spectacle, he usually goes to an eye specialist in order to find out what kind of lenses is required for his eyes. The eye specialist measures e.g. the visual acuity to find out what lenses he needs to prescribe. Afterwards, the customer has to visit an optician to find a suitable spectacle frame for the prescribed lenses. At the optician, he has to choose one of the exhibited frames. After that, the lenses and the corresponding frame are ordered and the customer has to wait a few days and sometimes even a few weeks, until the custom-made spectacle has been produced. After manufacture of the spectacle, the customer has to visit the optician again, so that the optician can slightly adapt the spectacle to the face of the customer.

It is a big disadvantage of the described procedure that the customer has to visit the optician several times and that he has to wait for his spectacles a couple of days. Furthermore, only the lenses are really custom-made, whereas the frame is standard and does not perfectly fit to the face of the customer. The optician can adapt the finished spectacle only very slightly by deforming the frame sides and the bows. Another disadvantage is that suchlike standard frames are available only in a few different colors and shapes. Today, the customer has no possibility to freely determine an individual shape or color of the frame.

### SUMMARY

It is therefore an object of the present invention to provide a device and a method for producing custom-made spectacles comparatively fast and cost effective, wherein also the frame of the spectacle can be individually designed by the customer and individually adapted to the customer's face.

The object of the present invention can be achieved with a device for producing custom-made spectacles having a scanning unit and a producing unit, wherein the scanning unit is configured for scanning at least a part of a customer's face and wherein the producing unit comprises at least a printing device for printing a spectacle lens and/or a spectacle frame, wherein the printing device is configured for printing the spectacle lens and/or the spectacle frame in dependency of scanning data of the scanning unit.

It is herewith advantageously possible to produce a spectacle in a very short time whose lenses and/or frames are individually adapted to the face of the customer by printing the spectacle lenses and/or the spectacle frame. It is conceivable that the production of the spectacle can be finished only a few minutes after the customer has been scanned because the overall production time mainly depends on the printing time. Although the produced spectacle is highly individual and custom-made, the customer can use the spectacle preferably immediately after ordering. In particular, several appointments are not necessary. Furthermore, the design, the shape, the dimensions, the color and the like can freely be determined by the customer because a printing process offers a high degree of freedom when designing the frame. In particular, the customer is no longer limited to only a few different embodiments of certain standard spectacle frames. The device can be fixed, e.g. at an eye specialist, an optician, a spectacle store, a department store or the like, or portable for temporary use in a store, at a customer's home or in a developing country, for instance. A spectacle in the sense of the present invention is in particular any kind of visual equipment having at least an optical lens. The spectacle comprises an eyeglass, an eyepiece, an ocular, a stereoscopy spectacle (with colored lenses or with lenticular structures onto the lens), a monocle, a frame-less and flat spectacle, a loupe and/or a binoculars.

According to a preferred embodiment of the present invention, the scanning unit comprises an eye scanner for scanning the customer's eyes in order to determine the visual capacity of the customer's eyes. The eye scanner comprises e.g. a refractometer, optometer and/or an ophthalmoscope (objective determination method). The determined parameters regarding the visual capacity of the customer are hereinafter referred to as visual capacity parameters. It is also conceivable that the device comprises a test bench for manually determining the visual capacity of the customer by eye examination (subjective determination method). The customer has to look onto a display illustrating an eye chart. The eye chart is subsequently modified and the user has to enter whether the illustrated signs on the eye chart could be identified clearly or not. Preferably, the customer has to look through an optic which contains lenses of different strengths that can be moved into his view. The device asks the customer if the chart appears more or less clear when different lenses are in place in order to determine the customer's visual capacity.

Alternatively or additionally, the scanning unit comprises a 3D scanner, e.g. a laser scanner or a scanner with multiple optical CCD (Charged-Coupled Device) cameras, for determining the three-dimensional shape of the customer's face or even the prow file of the whole customer's head. The knowledge of the individual shape of the customer's face advantageously allows to perfectly adapt the form of the spectacle to the customer's face. In this way, the wearing comfort for the customer can be increased significantly. The parameters determined by the scanning unit are hereinafter referred to as scanning parameters.

According to another preferred embodiment of the present invention, the device comprises a graphical user interface. It is herewith advantageously possible that the customer selects a certain base frame, which preferably has already been adapted to the shape of the customer's face, and subsequently freely modifies the design of the base frame to his individual requests and requirements. For example, the color, the pattern, the shape, the thickness of the frame is freely configured by the customer. Suchlike individually configurable parameters are referred to as customer parameters. In particular, the user interface comprises a display and preferably a touch pad which displays the actual design of the spectacle. Preferably, the device comprises a memory unit for storing the completed spectacle design, so that the individually designed spectacle can be reordered by the customer via internet at a later date, for instance.

According to another preferred embodiment of the present invention, the device comprises a processing unit for collecting the scanning data, the customer parameters and/or the visual capacity parameters and for calculating printing parameters in dependency of the scanning data, the customer parameters and/or the visual capacity parameters. The printing parameters are selected in such a manner that the printer provided only with the printing parameters prints a spectacle in accordance with the individual wishes of the customer. The printer comprises e.g. an inkjet printer or a 3D printer having several printing ink reservoirs for different types of printing ink and at least one curing device, e.g. an UV LED. One printing ink reservoir is filled with transparent printing ink and at least one other printing ink reservoir is filled with colored printing ink. The inkjet printer is capable of printing transparent printing ink for building up the spectacle lens and printing the colored printing ink for building up the spectacle frame. Preferably, the printer comprises multiple printing ink reservoirs which are filled with different colors, so that almost every color can be mixed for building up a frame in a desired special color. Furthermore, the transparent ink can be mixed with colored ink in order to build up colored lenses, e.g. to produce sunglasses or more trendy glasses. Preferably, the printer further comprises an ink reservoir for a coating ink, so that the printed lenses and/or the printed frame can be coated with a top-coat e.g. to increase the scratching resistance or for anti-reflection purposes. According to another preferred embodiment of the present invention, the device comprises an electronic cash terminal for payment of the spectacle by the customer. It is herewith advantageously possible that the payment process is performed electronically. Preferably, the device automatically calculates the actual costs for the printed spectacle and the customer pays the costs with his cash card or credit card.

Another subject of the present invention is a method for producing custom-made spectacles comprising the steps of scanning at least a part of a customer's face and generating corresponding scanning data in a first step and printing a spectacle lens and/or a spectacle frame in dependency of the scanning data in a second step. It is herewith advantageously possible to produce the custom-made spectacle very fast and cost-effective compared to the prior art. Furthermore, the frame of the spectacle can be individually designed by the customer and individually adapted to the customer's face, as described above.

According to a preferred embodiment of the present invention, the customer's eyes are scanned and scanning data comprising information about the customer's eyes are generated in the first step and/or wherein the shape and/or dimensions of the customer's face are scanned and scanning data comprising information about the shape and/or dimensions of the customer's face are generated in the first step. Preferably, manually entered individual customer wishes are detected in a third step and further processed as customer processed. Furthermore, it is conceivable that the customer's visual capacity is determined in a subject eye test in a fourth step. Here, the customer is e.g. provided with an eye chart illustrated on a display of the device and has to enter if he could identify the illustrated signs on the eye chart clearly or not. Subsequently, the device determines the visual capacity of the customer's eyes in dependency of the feedback information entered by the customer.

According to another preferred embodiment of the present invention, an individual design of a spectacle is calculated in dependency of the scanning data, the customer parameters and/or the visual capacity parameters in a fifth step and printing parameters are determined representing the calculated design in a sixth step, wherein the spectacle lens and/or the spectacle frame is printed in dependency of the printing parameters in the second step.

According to another preferred embodiment of the present invention, the spectacle lens is produced by depositing multiple droplets of a transparent printing ink at least partially on top of each other and/or side by side in the second step, wherein the spectacle frame is produced by depositing multiple droplets of colored printing inks at least partially on top of each other and/or side by side in the second step. Particularly, each droplet is cured by UV light after depositing. It is conceivable that the droplets are printed onto a substrate, wherein the finished spectacle has to be manually detached from the substrate.

These and other characteristics, features and advantages of the present invention will become apparent from the following detailed description, taken in conjunction with the accompanying drawings, which illustrate, by way of example, the principles of the invention. The description is given for the sake of example only, without limiting the scope of the invention. The reference figures quoted below refer to the attached drawings.

### BRIEF DESCRIPTION OF THE DRAWINGS

FIG. 1 illustrates schematically a device for producing custom-made spectacles according to an exemplary embodiment of the present invention.

FIG. 2 illustrates schematically a method for producing custom-made spectacles according to the exemplary embodiment of the present invention.

### DETAILED DESCRIPTION

The present invention will be described with respect to particular embodiments and with reference to certain drawings but the invention is not limited thereto but only by the claims. The drawings describe the invention only schematically and non-limiting. In the drawings, the size of some of the elements may be exaggerated and not drawn on scale for illustrative purposes.

Where an indefinite or definite article is used when referring to a singular noun, e.g. “a”, “an”, “the”, this includes a plural of that noun unless something else is specifically stated.

Furthermore, the terms first, second, third and the like in the description and in the claims are used for distinguishing between similar elements and not necessarily for describing a sequential or chronological order. It is to be understood that the terms so used are interchangeable under appropriate circumstances and that the embodiments of the invention described herein are capable of operation in other sequences than described of illustrated herein.

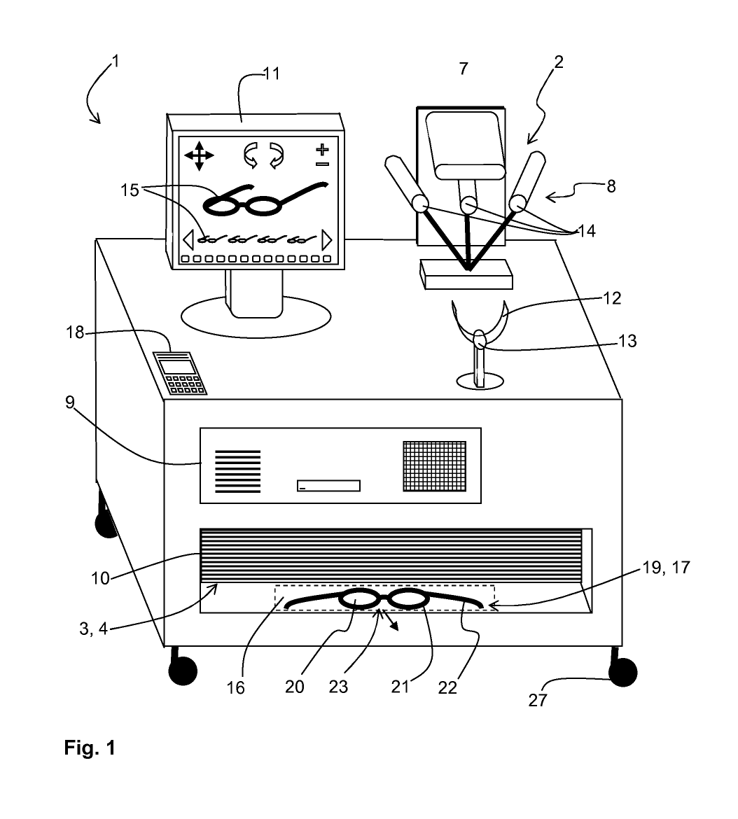

In FIG. 1, a device 1 for producing a custom-made spectacle 19 according to an exemplary embodiment of the present invention is shown. The device 1 is designed to produce a spectacle 19 for a customer (not shown) whose lenses 20 and frame 21 are individually adapted to the shape and the dimensions of the customer's face in order to increase the wearing comfort for the customer. The spectacle frame 21 is individually designed to the design wishes of the customer, wherein the spectacle lenses 20 are specially tailored regarding the visual capacity of the customer.

For this purpose, the device 1 comprises a scanning unit 2 for scanning the customer's face. The scanning unit 2 comprises an eye scanner 7 and a 3D scanner 8. The eye scanner 7 is provided for automatically determining the visual capacity of the customer's eyes by an objective determination method. The device 1 comprises a positioning means 12 with a positioning surface 13, on which the customer has to position his chin. Afterwards, the eye scanner 7 is capable of scanning the customer's eyes, wherein the refractive error of the customer's eyes is measured with refractometer or optometer means. The measured information's are forwarded as visual capacity parameters 24 to a processing unit 9 of the device 1. The processing unit 9 analyses the visual capacity parameters 24 and determines the specification of the lenses required to compensate the measured refractive errors.

Alternatively or additionally, the scanning unit comprises a 3D scanner, e.g. a laser scanner or a scanner with multiple optical CCD (Charged-Coupled Device) cameras 14, for determining the three-dimensional shape and the dimensions of the customer's face. The measured information's are forwarded as scanning parameters 6 to the processing unit 9. The processing unit 9 analyses the scanning parameters 6 and determines the required shape and dimensions of the spectacle frame 21 and the spectacles lenses 20 to ensure that the spectacle 19 will fit perfectly to the customer's face later on.

The device 1 further comprises a graphical user interface 11 which, in the present example, is a touch screen. The touch screen is controlled by the processing unit 9 and displays a multitude of different base frames 15 as proposals and point of departure for the customer. The customer has the possibility of viewing the different base frames 15 and to select one of the different base frames 15. Afterwards, the processing unit 9 roughly modifies the selected base frame 15 in dependency of the scanning parameters 6, so that the modified base frame 15 would fit to the customers' face. Now, the customer can freely design the modified frame 15 in according with his design requirements and individual wishes. For example, parameters, like e.g. color, pattern, transparency, brilliance, form, thickness, height and width of the frame, can be freely varied. Every step of modifying the displayed spectacle 15 by selections of the customer is taken into account by the processing unit 9, so that the touch screen always shows the actual version of the modified spectacle 15. Afterwards, the customer can select different types of spectacle lenses. Also, the color of the lenses 20 can be individually configured by the customer, in particular, when the spectacle should become a sunglass. Furthermore, the customer can select an anti-scratch coating or anti-reflection coating. After selecting and modifying the desired lenses 20, the processing unit 9 calculates the required lens parameters for the customer in dependency of the visual capacity parameters 24 and modifies the selected lenses 20 accordingly. The spectacle frame 21 is combined with these spectacle lenses 20 on the touch screen in order to illustrate the complete spectacle 15. It is conceivable that the actual version of the illustrated spectacle 15 is combined with a picture or even a 3D model of the customers face generated by the scanning unit 8 during the foregoing scanning step, so that the customer gets a real impression how he would look like when wearing the planned and individually designed spectacle. The frame and lens parameters individually configured by the customer are referred to as customer parameters 5. After the customer finishes the design of the spectacle 19, the processing unit 9 prepares printing parameters 25 for a production unit 3 of the device 1, wherein the final design of the whole spectacle 19 is completely determined by the printing parameters 25.

The production unit 3 of the device comprises an inkjet printer 10 as printing device 4 having a movable print head for depositing droplets of a printing ink onto a substrate 16, an UV curing means for curing droplets deposited onto the substrate 16 and several printing ink reservoirs for providing the print head with different printing inks.

Preferably, the printing procedure is accomplished by a method of printing a device as disclosed in the international patent application WO 2010/091 888 A1 which is incorporated herewith by reference. The printer 10 and the print head of the printer 10 are preferably designed like the printer and the print head disclosed in the European patent application EP 2 392 473 A1 which is herewith incorporated by reference.

In principle, the printing parameters 25 comprise information on how printing ink should be locating onto the substrate 16. For each droplet of printing ink to be deposited onto the substrate 16, the precise position onto the substrate 16, the kind of printing ink (what reservoir should be used), the size of the droplet (amount of printing ink), the curing intensity or curing time is determined by the printing parameters 25. The formation of the three dimensional spectacle is achieved by depositing a huge number of single droplets at least partially above and beside each other. The printing of the lenses 20 are performed by printing mostly droplets of transparent printing ink, wherein a non-transparent frame 21 is produced by printing mainly droplets of colored printing ink. Preferably, the printer comprises multiple printing ink reservoirs which are filled with different colors, so that almost every desired color can be mixed up by depositing droplets of different colors onto the substrate. If the time until the droplets are cured by the curing means is increased, the droplets of different colors merge with each other, so that a uniform or a precisely defined flow of color can be achieved. Alternatively, the different colors are mixed in a pre-mix chamber of the printer in order to achieve a certain color. Furthermore, the transparent ink can be mixed with colored ink in order to build up colored lenses, e.g. to produce sunglasses or more trendy glasses. Preferably, the printer further comprises an ink reservoir for a coating ink, so that the printed lenses and/or the printed frame can be coated with a top-coat e.g. to increase the scratching resistance or for antireflection purposes.

When the printing of the spectacle 19 is finished, the spectacle 19 can manually be detached from the substrate 16. The production of the spectacle 19 is completed by folding the frame sides 22 backwards by approximately 90 degree. It is alternatively also conceivable that the production of the spectacle 19 is completed by mounting manually a hinge joint between the frame front 23 and the respective frame sides 22.

According to a preferred embodiment, the device 1 is provided with a test bench (not shown) for determining the visual capacity of the customer. In this case, the customer has to look onto a display illustrating an eye chart for testing purposes. The eye chart is subsequently modified and the customer answers questions on the touch pad whether the illustrated signs on the eye chart could be seen clearly, more clearly, less clearly or not at all by the customer. The processing unit 9 calculates the visual capacity parameters in dependency of the customer's answers in view of the corresponding eye charts. Preferably, the customer has to look through an optic which contains lenses of different strengths that can be moved into his view. The device asks the customer if the chart appears more or less clear when different lenses are in place.

Preferably, the device 1 comprises an electronic cash terminal 18 for payment of the spectacle by the customer. Preferably, the processing unit 8 calculates the actual costs for the printed spectacle 19 automatically and the customer pays the costs with his cash card or credit card.

It is conceivable that the device 1 works perfectly independently, so that the customer does not need any kind of a human customer consultant for generating, designing, producing and buying his custom-made spectacle 19.

Preferably, the device 1 is provided with wheels 27 on the bottom side of the device 1, so that the device 1 is portable.

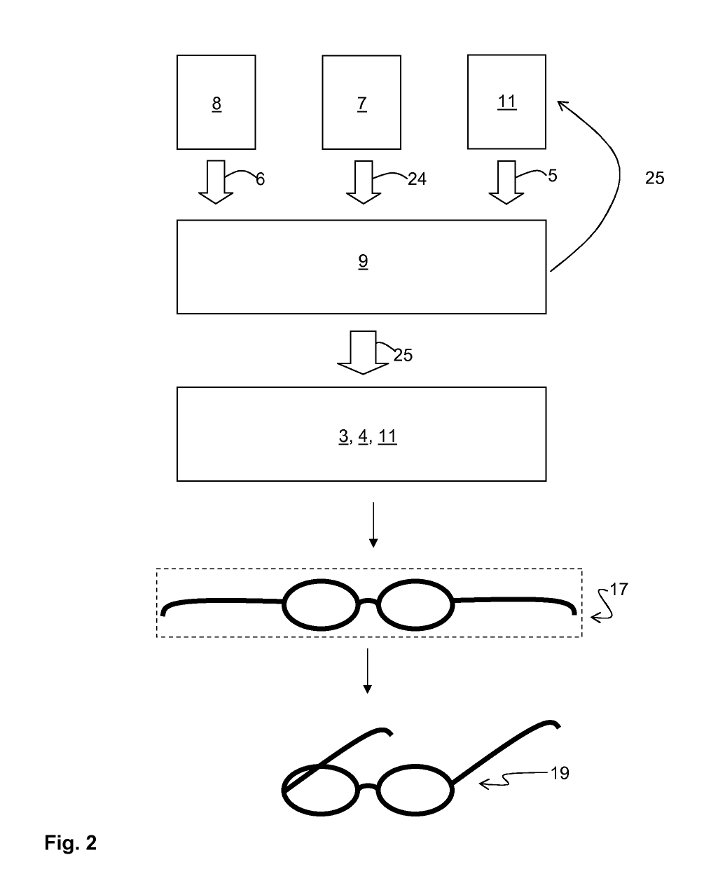

In FIG. 2 a method for producing custom-made spectacles 19 according to the exemplary embodiment of the present invention is schematically illustrated. In the first step, the scanning parameters 6 are generated through scanning of the customer's face by the 3D scanner 8 of the scanning unit 2. Furthermore, the customer's eyes are scanned by the eye scanner 7 of the scanning unit 2 in order to generate the visual capacity parameters 3. The customer designs the spectacle 19 by entering his individual requirements and requests via the user interface 11 in a third step. The information regarding the customer's individual requests and requirements are processed as the customer parameters 5. The processing unit 9 calculates the individual design of the spectacle 19 to be printed in dependency of the scanning parameters 6, the visual capacity parameters 24 and the customer parameters 5. Preferably, display parameters 26 which correspond to the actual design of the spectacle 19 are sent to the graphical user interface 11 in order to display the actual design to the customer. Furthermore, the processing unit 9 calculates printing parameters 26 from the actual design of the spectacle 19 and transmits the printing parameters 26 to the printing device 4. The printing device 4 comprises the inkjet printer 11 for printing the spectacle frame 22 and the spectacle lenses 20 onto the substrate 16. After the printed product 17 has been delivered from the printer 11, the spectacle 19 is detached manually from the substrate 16 and the frame sides 22 are folded backwards by approximately 90 degree in order to finish the production of the spectacle 19.

### LIST OF REFERENCE SIGNS

1 device
2 scanning unit
3 production unit
4 printing device
5 customer parameters
6 scanning parameters
7 eye scanner
8 3D scanner
9 processing unit
10 inkjet printer
11 graphical user interface
12 positioning means
13 positioning surface
14 CCD camera
15 base frame
16 substrate
17 printed product
18 electronic cash terminal
19 spectacle
20 lens
21 frame
22 frame sides
23 frame front
24 visual capacity parameters
25 printing parameters
26 display parameters
27 wheels

What claimed is:

1. A device for producing custom-made spectacles comprising:
a scanning unit and
a producing unit,
wherein the scanning unit is configured for scanning at least a part of a customer's face,
wherein the producing unit comprises at least a printing device for printing a spectacle lens and/or a spectacle frame,
wherein the printing device comprises an inkjet printer having an ink reservoirs for transparent printing ink and multiple ink reservoirs for printing ink of different color,
wherein the scanning unit comprises a 3D scanner for scanning he shape and/or dimensions of the customer's face,
wherein the device comprises a graphical user interface for manually entering individual customer parameters,
wherein the printing device is configured for printing the spectacle lens and/or the spectacle frame in dependency of the customer parameters,
wherein the device comprises a processing unit for determining individual printing parameters for the printing device in dependency of scanning parameters, customer parameters and/or visual capacity parameters to adapt the spectacle to the customer's face and to the customer's demands,
wherein the printing device is configured for printing the spectacle lens and/or the spectacle frame in dependency of scanning data of the scanning unit.

2. The device according to claim 1, wherein the scanning unit comprises an eye scanner for scanning the customer's eyes.

3. (canceled)

4. The device according to claim 1, wherein the device comprises a test bench for determining the visual capacity of the customer.

5-15. (canceled)

16. The device according to claim 1, wherein the inkjet printer is capable of printing transparent printing ink for building up the spectacle lens and
wherein the inkjet printer is capable of printing colored printing ink for building up the spectacle frame.

17. The device according to claim 1, wherein the inkjet printer comprises an ink reservoir for a coating ink and
wherein the inkjet printer is capable of coating the spectacle lens and/or the spectacle frame with the coating ink.

18. The device according to claim 1, wherein the device comprises an electronic cash terminal for payment of the spectacle by the customer.

19. A method for producing custom-made spectacles comprising the following steps:
scanning at least a part of a customer's face and generating corresponding scanning data in a first step and;
printing a spectacle lens and/or a spectacle frame in dependency of the scanning data in a second step.

20. The method according to claim 19, wherein the customer's eyes are scanned and scanning data comprising information about the customer's eyes are generated in the first step and/or wherein the shape and/or dimensions of the customer's face are scanned and scanning data comprising information about the shape and/or dimensions of the customer's face are generated in the first step.

21. The method according to claim 19, wherein manually entered individual customer parameters are detected in a third step and/or wherein the customer's visual capacity is determined in an eye test in a fourth step.

22. The method according to claim 19, wherein an individual design of a spectacle is calculated in dependency of scanning parameters, the customer parameters and/or the visual capacity parameters in a fifth step and printing parameters are determined representing the calculated design in a sixth step, wherein the spectacle lens and/or the spectacle frame is printed in dependency of the printing parameters in the second step.

23. The method according to claim 19, wherein the spectacle lens is produced by depositing multiple droplets of a transparent printing ink at least partially on top of each other and/or side by side in the second step.

24. The method according to claim 19, wherein the spectacle frame is produced by depositing multiple droplets of colored printing inks at least partially on top of each other and/or side by side in the second step.

25. The device according to claim 2, wherein the device comprises a test bench for determining the visual capacity of the customer.

26. The device according to claim 2, wherein the inkjet printer is capable of printing transparent printing ink for building up the spectacle lens and
wherein the inkjet printer is capable of printing colored printing ink for building up the spectacle frame.

27. The device according to claim 25, wherein the inkjet printer is capable of printing transparent printing ink for building up the spectacle lens and
wherein the inkjet printer is capable of printing colored printing ink for building up the spectacle frame.

28. The device according to claim 2, wherein the device comprises an electron cash terminal for payment of the spectacle by the customer.

29. The device according to claim 27, wherein the device comprises an electronic cash terminal for payment of the spectacle by the customer.

30. The method according to claim 20, wherein manually entered individual customer parameters are detected in a third step and/or wherein the customer's visual capacity is determined in an eye test in a fourth step.

31. The method according to claim 20, wherein an individual design of a spectacle is calculated in dependency of scanning parameters, the customer parameters and/or the visual capacity parameters in a fifth step and printing parameters are determined representing the calculated design in a sixth step, wherein the spectacle lens and/or the spectacle frame is printed in dependency of the printing parameters in the second step.

32. The method according to claim 31, wherein the spectacle lens is produced by depositing multiple droplets of a transparent printing ink at least partially on top of each other and/or side by side in the second step.
